(19) United States
(12) Patent Application Publication
(10) Pub. No.: US 2006/0241574 A1
(43) Pub. Date: Oct. 26, 2006

(54) Electromagnetic energy distributions for electromagnetically induced disruptive cutting

(76) Inventor: Ioana M. Rizoiu, San Clemente, CA (US)

Correspondence Address: STOUT, UXA, BUYAN & MULLINS LLP, 4 VENTURE, SUITE 300, IRVINE, CA, 92618, US

(21) Appl. No.: 11/033,032

(22) Filed: Jan. 11, 2005

Related U.S. Application Data
(63) Continuation-in-part of application No. 10/993,498, filed on Nov. 18, 2004.
Application No. 10/993,498 is a continuation of application No. 10/164,451, filed on Jun. 6, 2002, now Pat. No. 6,821,272.
Application No. 10/164,451 is a continuation of application No. 09/883,607, filed on Jun. 18, 2001, now abandoned.
Application No. 09/883,607 is a continuation of application No. 08/903,187, filed on Jun. 12, 1997, now Pat. No. 6,288,499.
Application No. 08/903,187 is a continuation-in-part of application No. 08/522,503, filed on Aug. 31, 1995, now Pat. No. 5,741,247.

(51) Int. Cl. A61B 18/18

(52) U.S. Cl. 606/10

(57) ABSTRACT

Output optical energy pulses including relatively high energy magnitudes and steep slope at the beginning of each pulse are disclosed. As a result of the relatively high energy magnitudes which lead each pulse, the leading edge of each pulse includes a relatively steep slope. This slope is preferably greater than or equal to 5. Additionally, the full-width half-max value of the output optical energy distributions are between 0.025 and 250 microseconds and, more preferably, are about 50-70 microseconds. A flashlamp is used to drive the laser system, and a current is used to drive the flashlamp. A flashlamp current generating circuit includes a solid core inductor which has an inductance of about 50 microhenries and a capacitor which has a capacitance of about 50 microfarads. The output optical energy pulses cut target surfaces by interacting with fluid that is located above, on and/or in the target surface. Methods are disclosed for therapeutically treating tissue with pulses of electromagnetic energy.

### CROSS-REFERENCE TO RELATED APPLICATIONS

This application claims the benefit of U.S. Provisional Application No. 60/535,004, filed Jan. 8, 2004, the contents of which are expressly incorporated herein by reference. This application is also a continuation-in part application of U.S. application Ser. No. 10/993,498, filed Nov. 18, 2004 and entitled ELECTROMAGNETIC ENERGY DISTRIBUTIONS FOR ELECTROMAGNETICALLY INDUCED MECHANICAL CUTTING, which is a continuation application of U.S. application Ser. No. 10/164,451, filed Jun. 6, 2002 and entitled ELECTROMAGNETIC ENERGY DISTRIBUTIONS FOR ELECTROMAGNETICALLY INDUCED MECHANICAL CUTTING, which is a continuation application of U.S. application Ser. No. 09/883,607, filed Jun. 18, 2001 and entitled ELECTROMAGNETIC ENERGY DISTRIBUTIONS FOR ELECTROMAGNETICALLY INDUCED MECHANICAL CUTTING, which is a continuation application of U.S. application Ser. No. 08/903,187, filed Jun. 12, 1997, now U.S. Pat. No. 6,288,499 and entitled ELECTROMAGNETIC ENERGY DISTRIBUTIONS FOR ELECTROMAGNETICALLY INDUCED MECHANICAL CUTTING, which is a continuation-in-part of U.S. application Ser. No. 08/522,503, filed Aug. 31, 1995 and entitled ATOMIZED FLUID PARTICLES FOR ELECTROMAGNETICALLY INDUCED CUTTING, now U.S. Pat. No. 5,741,247, all of which are commonly assigned and the contents of which are expressly incorporated herein by reference.

### BACKGROUND OF THE INVENTION

1. Field of the Invention

The present invention relates generally to electronic devices and, more particularly, to output optical energy distributions of lasers.

2. Description of Related Art

A variety of electromagnetic laser energy generating architectures have existed in the prior art. A solid-state laser system, for example, generally comprises a laser rod for emitting coherent light and a source for stimulating the laser rod to emit the coherent light. Flashlamps are typically used as stimulation sources for middle infrared lasers between 2.5 μm and 3.5 μm, such as Er,Cr:YSGG and Er:YAG laser systems, for example. The flashlamp is driven by a flashlamp current, which comprises a predetermined pulse shape and a predetermined frequency.

The flashlamp current drives the flashlamp at the predetermined frequency, to thereby produce an output flashlamp light distribution having substantially the same frequency as the flashlamp current. This output flashlamp light distribution from the flashlamp drives the laser rod to produce coherent light at substantially the same predetermined frequency as the flashlamp current. The coherent light generated by the laser rod has an output optical energy distribution over time that generally corresponds to the pulse shape of the flashlamp current.

The pulse shape of the output optical energy distribution over time typically comprises a relatively gradually rising energy that ramps up to a maximum energy, and a subsequent decreasing energy over time. The pulse shape of a typical output optical energy distribution can provide a relatively efficient operation of the laser system, which corresponds to a relatively high ratio of average output optical energy to average power inputted into the laser system.

The prior art pulse shape may be suitable for cutting procedures, for example, where the output optical energy is directed onto a target surface to induce cutting of the contact tissue. However, when thermal cutting is employed utilizing certain conventional procedures, undesirable secondary damage, such as charring or burning of surrounding structures or tissues, may occur. Newer cutting procedures, however, may not altogether rely on laser-induced thermal heating only. More particularly, a cutting mechanism, such as that disclosed in U.S. Pat. No. 5,741,247, directs output optical energy from a laser system first into a distribution of atomized fluid particles located in a volume of space above the target surface. Disruptive (e.g., mechanical, thermo-mechanical, and other) cutting forces then can be imparted onto the tissue. In certain implementations, at least a portion of the output optical energy interacts with the atomized fluid particles, causing the atomized fluid particles to expand, wherein electromagnetically-induced disruptive forces may be imparted onto the target surface. As a result of the unique interactions of the output optical energy with the atomized fluid particles, many prior art output optical energy distribution pulse shapes and frequencies have not been especially suited for providing optimal electromagnetically-induced disruptive (e.g., mechanical, thermo-mechanical, and other) processes such as for example cutting, removing, ablating, cleaning and others. Specialized output optical energy distributions may be advantageous for optimal cutting, for example, when the output optical energy is directed into a distribution of atomized fluid particles for effectuating a transfer of pulse energy that is initially coupled into the highly absorbing molecules of the atomized fluid particles and secondly into the highly absorbing molecules of the material to be cut.

### SUMMARY OF THE INVENTION

The output optical energy distributions disclosed herein comprise relatively high energy spiking with a relatively steep leading edge at the beginning of each pulse. The slope of the pulse or pulses is preferably greater than or equal to 5. Additionally, the full-width half-max (FWHM) values of the output optical energy distributions are greater than 0.025 microseconds. More preferably, the full-width half-max values are between 0.025 and 250 microseconds and, more preferably, are between 10 and 150 microseconds. The full-width half-max value of about 70 microseconds is in the illustrated embodiment. A flashlamp is used to drive the laser system, and a current is used to drive the flashlamp. A flashlamp current generating circuit comprises a solid core inductor having an inductance in a range of about 30 to about 70 microhenries and a capacitor having a capacitance in a range of about 30 to about −70 microfarads.

The output optical energy distributions disclosed herein permit a cutting apparatus to cut a target surface, such as body tissue, with reduced, and preferably no, undesirable secondary damage to the target surface. The apparatus may cut the target surface without requiring application of additional fluids, or in other words, the cutting of the target tissue may occur by thermal energy of the output energy alone, or in combination, with disruptive (e.g., mechanical, thermo-mechanical and other) energy imparted by or in connection with disruption of fluid particles located above the target surface, on the target surface, or within the target surface. Output optical energy from a laser system can be directed first into a distribution of atomized fluid particles located in a volume of space just above the target surface, and then into the material wherein absorbing molecules are exposed to very fast rising pulses with a steep slope, causing a localized expansion of that component of the material and subsequent removal of that material with, in some embodiments, minimal to no thermal heat deposition into the material. The apparatus may also include a filter to spatially and temporally modify electromagnetic energy transmitted from the electromagnetic energy source. The filter may comprise a fluid, such as water, and may be provided as a distribution of atomized fluid particles.

The present invention further may comprise a method of remodeling tissue. According to an implementation of the method, pulses of electromagnetic energy are directed toward a surface of the tissue, and the pulses can be adjusted to achieve localized melting and/or reforming of the target surface and/or tissue.

As another aspect of the present invention, a method of delivering ions to a target surface is disclosed. According to this aspect, particles may be projected onto the target surface, and the surface, with the embedded ions or ions that have been mechanically retained within the surface, may be remodeled.

The present invention, together with additional features and advantages thereof, may best be understood by reference to the following description taken in connection with the accompanying illustrative drawings.

### BRIEF DESCRIPTION OF THE DRAWINGS

FIG. 1 is a plot of flashlamp-driving current versus time according to the prior art;

FIG. 2 is a plot of output optical energy versus time for a laser system according to the prior art;

FIG. 3 is a schematic circuit diagram illustrating a circuit for generating a flashlamp-driving current in accordance with the present invention;

FIG. 4 is a plot of flashlamp-driving current versus time in accordance with the present invention;

FIG. 5 is a plot of output optical energy versus time for a laser system in accordance with the present invention;

FIG. 6 is a plot of a sequence of short and long pulses;

FIG. 7 is a magnified view of a short pulse shown in FIG. 6;

FIG. 8 is a magnified view of a long pulse shown in FIG. 6;

FIG. 9 is another magnified view of a short pulse shown in FIG. 6;

FIG. 10 is another magnified view of a long pulse shown in FIG. 6;

FIG. 11 is a partial flow diagram describing an implementation of a method of remodeling tissue according to the present invention; and

FIG. 12 is a partial flow diagram illustrating an implementation of a method of delivering ions to a target surface according to the present invention.

### DETAILED DESCRIPTION OF THE INVENTION

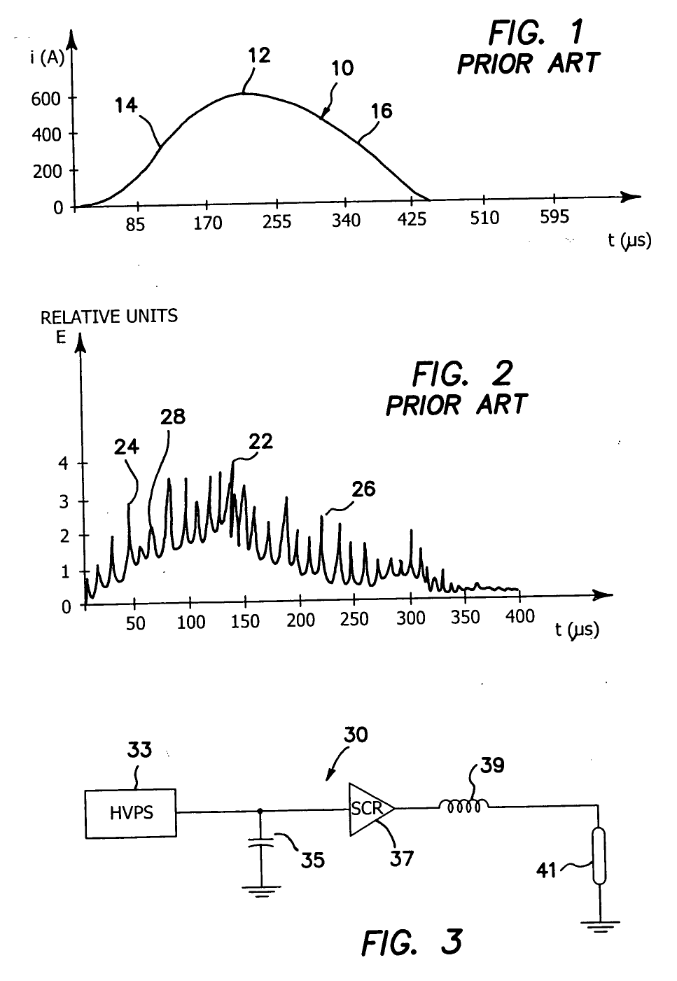

Referring more particularly to the drawings, FIG. 1 illustrates a plot of flashlamp-driving current versus time according to the prior art. The flashlamp-driving current 10 initially ramps up to a maximum value 12. The initial ramp 14 typically comprises a slope (current divided by time) of between 1 and 4. After reaching the maximum value 12, the flashlamp-driving current 10 declines with time, as illustrated by the declining current portion 16. Additionally, the flashlamp-driving current 10 of the prior art may typically comprise a pulse width of about 300 microseconds. The full-width half-max value of the flashlamp-driving current 10 is typically between 250 and 275 microseconds. The full-width half-max value is defined as a value of time corresponding to a length of the full-width at half-max range plotted on the time axis. The full-width half-max range is defined on the time axis from a beginning time, where the amplitude first reaches one half of the peak amplitude of the entire pulse, to an ending time, where the amplitude reaches one half of the peak amplitude a final time within the pulse. The full-width half-max value is the difference between the beginning time and the ending time.

FIG. 2 illustrates a plot of energy versus time for the output optical energy of a typical prior art laser. The output optical energy distribution 20 generally comprises a maximum value 22, an initial ramp 24, and a declining output energy portion 26. The micropulses 28 correspond to the oscillation relaxation process related to the change in population inversions within the laser rod as coherent light is generated by stimulated emission. The average power of the laser can be defined as the power delivered over a predetermined period of time, which typically comprises a number of pulses. The efficiency of the laser system can be defined as a ratio of the output optical power of the laser, to the input power into the system that is required to drive the flashlamp. Typical prior art laser systems are designed with flashlamp-driving currents 10 and output optical energy distributions 20 which optimize the efficiency of the system.

FIG. 3 illustrates an analog flashlamp-driving circuit 30 according to an embodiment of the present invention. The flashlamp-driving circuit 30 comprises a high-voltage power supply 33, a capacitor 35, a rectifier 37, an inductor 39, and a flashlamp 41. The capacitor 35 is connected between the high-voltage power supply 33 and ground, and the flashlamp 41 is connected between the inductor 39 and ground. The high-voltage power supply 33 preferably comprises a 1200 to 1500 volt source, having a charging rate of 1500 Joules per second. The flashlamp 41 may comprise a 450 to 900 torr source and, preferably, comprises a 700 torr source. The capacitor 35 comprises a 30 to 70 microfarad capacitor, and preferably a 50 microfarad capacitor, and the rectifier 37 preferably comprises a silicon-controlled rectifier. The inductor 39 comprises a 30 to 70 microhenry solid core inductor or equivalent, and preferably a 50 microhenry solid-core inductor or equivalent. In alternative embodiments, the inductor 39 may comprise a 13 microhenry inductance or between 10 and 15 micro-henries. In still other alternative embodiments, the inductor 39 may comprise inductance values of 13 microhenry inductance or between 10 and 15 microhenries in solid-core inductor or equivalent. To the extent practicable the circuit 30 may comprise digital components. Other values for the inductor 39 and the capacitance 35 may be implemented in order to obtain flashlamp-driving currents having relatively fast rising times, for example, as discussed below.

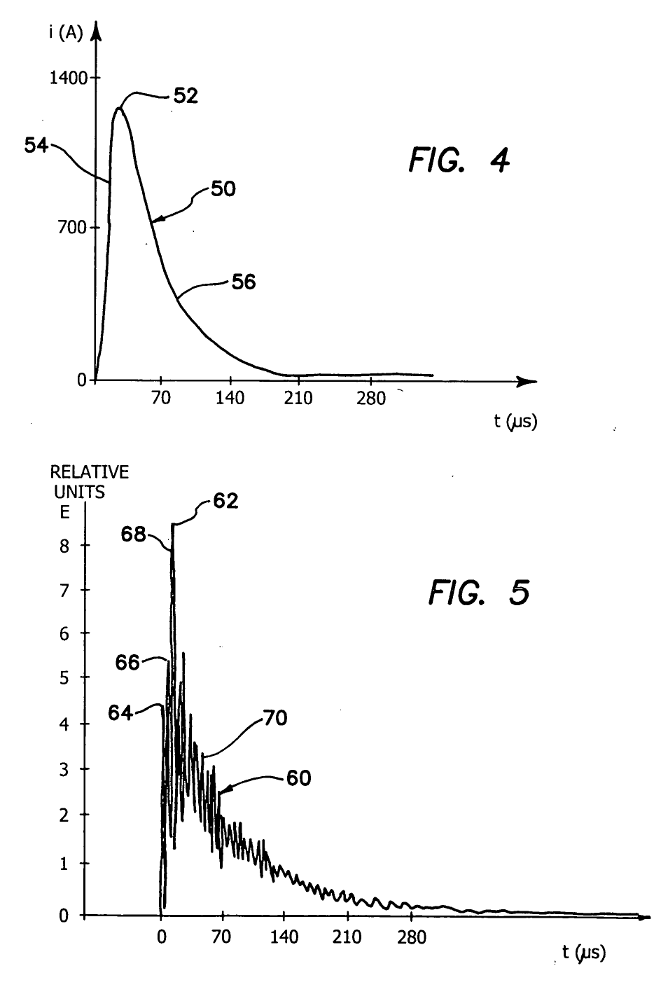

FIG. 4 illustrates the flashlamp driving current 50 of the present invention, which passes from the inductor 39 to the flashlamp 41. The flashlamp driving current of the present invention preferably has a pulse width which is greater than about 0.25 microseconds and, more preferably, which is in a range of 50 to 300 microseconds. In the illustrated embodiment, the pulse width is about 200 microseconds. The flashlamp driving current 50 comprises a maximum value 52, an initial ramp portion 54, and a declining current portion 56. The flashlamp 41 preferably comprises a cylindrical glass tube having an anode, a cathode, and a gas there between such as Xenon or Krypton. An ionizer circuit (not shown) ionizes the gas within the flashlamp 41. As the flashlamp-driving current 50 is applied to the anode of the flashlamp 41, the potential between the anode and the cathode increases. This potential increases as the flashlamp-driving current increases, as indicated by the initial ramp 54. Current flows through the gas of the flashlamp 41, resulting in the flashlamp 41 emitting bright incoherent light.

The flashlamp 41 can be close-coupled to, for example, a laser rod (not shown), which preferably comprises a cylindrical crystal. The flashlamp 41 and the laser rod are positioned parallel to one another with preferably less than 1 centimeter distance therebetween. The laser rod is suspended on two plates, and is not electrically connected to the flashlamp-driving current circuit 30. Although the flashlamp 41 comprises the preferred means of stimulating the laser rod, other means are also contemplated by the present invention. Diodes, for example, may be used instead of flashlamps for the excitation source. The use of diodes for generating light amplification by stimulated emission is discussed in the book Solid-State Laser Engineering, Fourth Extensively Revised and Updated Edition, by Walter Koechner, published in 1996, the contents of which are expressly incorporated herein by reference.

The incoherent light from the presently preferred flashlamp 41 impinges on the outer surface of the laser rod. As the incoherent light penetrates into the laser rod, atoms or ions within the laser rod absorb the penetrating light and subsequently emit coherent light through stimulation emission processes. The atoms or ions may comprise erbium and chromium, and the laser rod itself may comprise a crystal such as YSGG, for example. The presently preferred laser system comprises either an Er, Cr:YSGG solid state laser, which generates electromagnetic energy having a wavelength in a range of 2.70 to 2.80 microns, or an erbium, yttrium, aluminum garnet (Er:YAG) solid state laser, which generates electromagnetic energy having a wavelength of 2.940 microns. As presently preferred, the Er, Cr:YSGG solid state laser has a wavelength of approximately 2.789 microns and the Er:YAG solid state laser has a wavelength of approximately 2.940 microns. According to one alternative embodiment, the laser rod may comprises a YAG crystal, and the impurities may comprise erbium impurities. A variety of other possibilities exist, a few of which are set forth in the above-mentioned book Solid-State Laser Engineering, Fourth Extensively Revised and Updated Edition, by Walter Koechner, published in 1996, the contents of which are expressly incorporated herein by reference. Other possible laser systems include an erbium, yttrium, scandium, gallium garnet (Er:YSGG) solid state laser, which generates electromagnetic energy having a wavelength in a range of 2.70 to 2.80 microns; an erbium, yttrium, aluminum garnet (Er:YAG) solid state laser, which generates electromagnetic energy having a wavelength of 2.94 microns; chromium, thulium, erbium, yttrium, aluminum garnet (CTE:YAG) solid state laser, which generates electromagnetic energy having a wavelength of 2.69 microns; erbium, yttrium orthoaluminate (Er:YAL03) solid state laser, which generates electromagnetic energy having a wavelength in a range of 2.71 to 2.86 microns; holmium, yttrium, aluminum garnet (Ho:YAG) solid state laser, which generates electromagnetic energy having a wavelength of 2.10 microns; quadrupled neodymium, yttrium, aluminum garnet (quadrupled Nd:YAG) solid state laser, which generates electromagnetic energy having a wavelength of 266 nanometers; argon fluoride (ArF) excimer laser, which generates electromagnetic energy having a wavelength of 193 nanometers; xenon chloride (XeCl) excimer laser, which generates electromagnetic energy having a wavelength of 308 nanometers; krypton fluoride (KrF) excimer laser, which generates electromagnetic energy having a wavelength of 248 nanometers; and carbon dioxide (CO2), which generates electromagnetic energy having a wavelength in a range of 9 to 11 microns.

Particles, such as electrons, associated with the atoms or ions absorb energy from the impinging incoherent radiation and rise to higher valence states. The particles that rise to metastable levels remain at this level for periods of time until, for example, energy particles of the radiation excite stimulated transitions. The stimulation of a particle in the metastable level by an energy particle results in both of the particles decaying to a ground state and an emission of twin coherent photons (particles of energy). The twin coherent photons can resonate through the laser rod between mirrors at opposing ends of the laser rod, and can stimulate other particles on the metastable level, to thereby generate subsequent twin coherent photon emissions. This process is referred to as light amplification by stimulated emission of radiation.

The amplification effect will continue until a majority of particles, which were raised to the metastable level by the stimulating incoherent light from the flashlamp 41, have decayed back to the lower state. The decay of a majority of particles from the metastable state to the lower state results in the generation of a large number of photons, corresponding to an upwardly rising micropulse (64, for example, FIG. 5). As the particles on the ground level are again stimulated back up to the metastable state, the number of photons being emitted decreases, corresponding to a downward slope in the micropulse 64, for example. The micropulse continues to decline, corresponding to a decrease in the emission of coherent photons by the laser system. The number of particles stimulated to the metastable level increases to an amount where the stimulated emissions occur at a level sufficient to increase the number of coherent photons generated. As the generation of coherent photons increases, and particles on the metastable level decay, the number of coherent photons increases, corresponding to an upwardly rising micropulse.

The output optical energy distribution over time of the laser system is illustrated in FIG. 5 at 60. The output optical energy distribution of the present invention preferably has a pulse width that is greater than about 0.25 microseconds and, more preferably, in a range of 50 to 300 microseconds. In the illustrated embodiment, the pulse width is about 200 microseconds. The output optical energy distribution 60 comprises a maximum micropulse value 62, a number of leading micropulses 64, 66, 68, and a portion of generally declining optical energy 70.

According to the present invention, the output optical energy distribution 60 comprises a large magnitude. This large magnitude corresponds to one or more sharply-rising micropulses at the leading edge of the pulse. As illustrated in FIG. 5, the micropulse 68 comprises a maximum value 62 which is at or near the very beginning of the pulse. Additionally, the full-width half-max value of the output optical energy distribution in FIG. 5 is approximately 70 microseconds, and in other embodiments can be between about 40 and about 65 microseconds, compared to full-width half-max values of the prior art typically ranging from 250 to 300 microseconds. Applicant's invention contemplates pulses comprising full-width half-max values greater than 0.025 microseconds and, preferably, ranging from 10 to 150 microseconds, but other ranges may also be possible. Additionally, Applicant's invention contemplates a pulse width of between 0.25 and 300 microseconds, for example, compared to typical prior-art pulse widths which are greater than 300 microseconds. Another aspect of this invention is the combination of pulses from a pulse width range between 0.25 and 300 microseconds with pulses from a pulse width range between 300 microseconds and 800 microseconds. Further, a frequency of 1 to 20 Hz is presently preferred. Alternatively, frequencies of 20-150 Hz may be used. Applicants' invention generally contemplates frequencies between 1 and 150 Hz. In case of diode-pumped, Q-switched lasers, the frequencies may be as high as in the KHz range

As mentioned above, the full-width half-max range is defined from a beginning time, where the amplitude first rises above one-half the peak amplitude, to an ending time, where the amplitude falls below one-half the peak amplitude a final time during the pulse width. The full-width half-max value is defined as the difference between the beginning time and the ending time.

The location of the full-width half-max range along the time axis, relative to the pulse width, is closer to the beginning of the pulse than the end of the pulse. The location of the full-width half-max range is preferably within the first half of the pulse and, more preferably, is within about the first third of the pulse along the time axis. Other locations of the full-width half-max range are also possible in accordance with the present invention. The pulse rise time preferably occurs within the first 5 to 35 microseconds and, more preferably, occurs within the first 12.5 microseconds from the beginning of the pulse. The beginning time is preferably achieved within the first tenth of the pulse width.

Another distinguishing feature of the output optical energy distribution 70 is that the micropulses 64, 66, 68, for example, comprise approximately one-third of the maximum amplitude 62. More preferably, the leading micropulses 64, 66, 68 comprise an amplitude of approximately one-half of the maximum amplitude 62. In contrast, the leading micropulses of the prior art, as shown in FIG. 2, are relatively small in amplitude.

The slope of the output optical energy distribution 60 is greater than or equal to 5 and in another embodiment is greater than about 10. In the illustrated embodiment, the slope is about 50. In contrast, the slope of the output optical energy distribution 20 of the prior art is about 4.

In a further embodiment of the invention, such as embodiments in which the energy is used to improve cutting of soft tissues, the slope of the pulse may be less steep. For instance, the shape of the pulse may be smoother than the shapes discussed above. By utilizing pulses with less steep initial slopes it may be possible to achieve for example enhanced coagulation of the cut tissue.

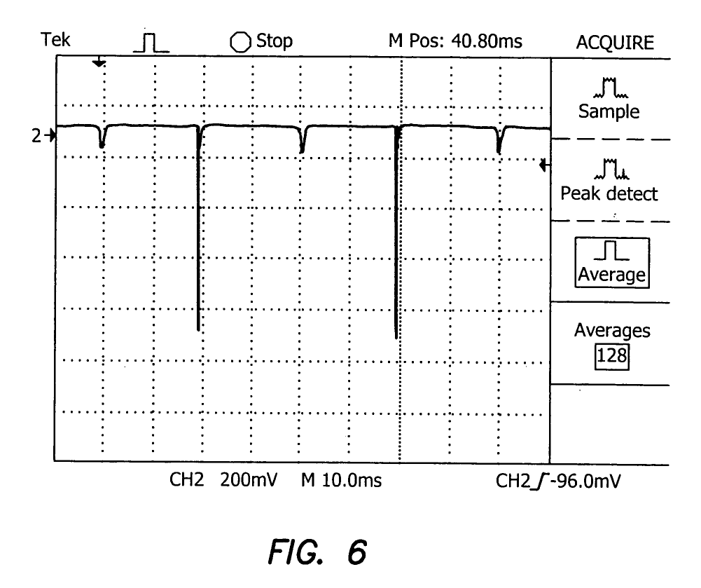

In certain embodiments, a cutting, coagulating and/or tissue re-modeling effect is achieved by alternating short and long pulses, such as shown in FIG. 6. In the illustrated embodiment of FIG. 6, the short pulses comprise relatively large amplitudes compared to the long pulses. For example, a cutting effect may be obtained by providing a train of pulses in various sequences of short and long pulses. In one embodiment, the train of pulses may include alternating short and long pulses. In another embodiment, the train of pulses may include a sequence of pulses such as long, long, short, or short, short, long. Additional patterns or sequences may also be utilized. By utilizing alternating or changing pulse shapes, it may be possible to obtain combined effects not achievable by any single pulse shape. For example, by utilizing a short pulse and long pulse combination, it may be possible to create a deep cut with a relatively strong coagulation. Typically, shorter pulses may tend to create a relatively deep cut with moderate coagulation, and longer pulses may tend to create a relatively shallow cut with strong coagulation. As used herein, a “long” pulse is a pulse that has a less steep slope and a longer tail compared to a shorter pulse. In certain embodiments, a long pulse can have a duration of about 700 microseconds.

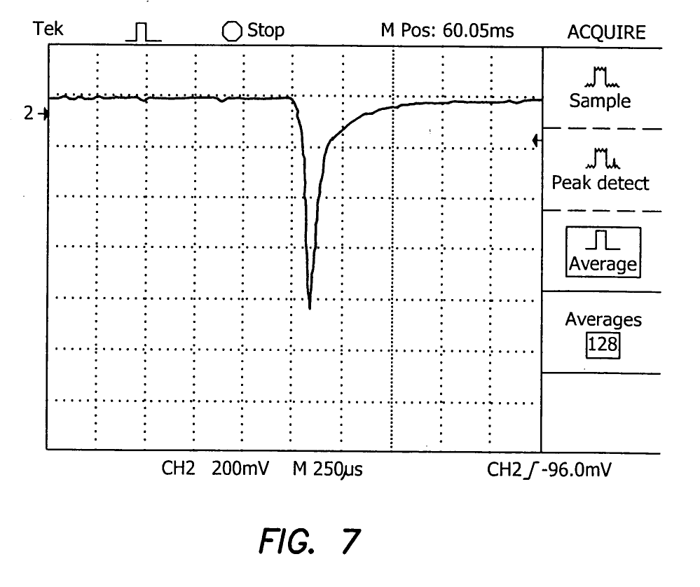

FIG. 7 illustrates a magnified view of a short pulse shown in FIG. 6. The pulse in FIG. 7 has a maximum amplitude of approximately 800 millivolts.

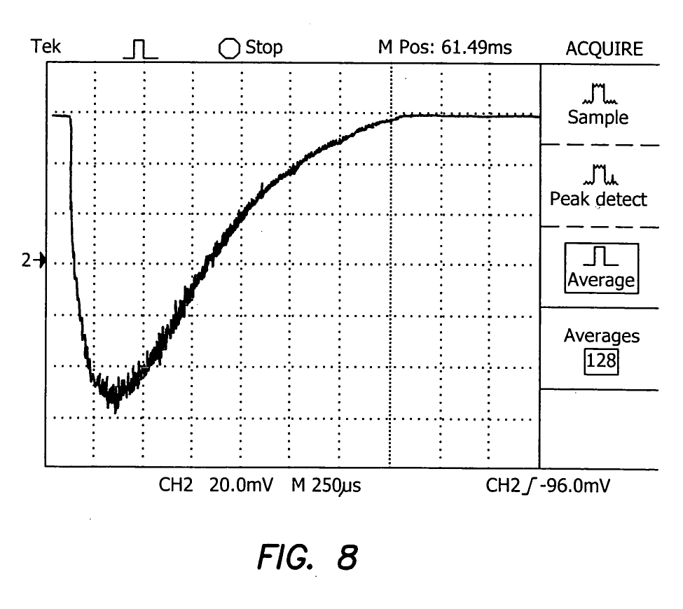

FIG. 8 illustrates a magnified view of a long pulse shown in FIG. 6. The pulse in FIG. 8 has a maximum amplitude of approximately 120 millivolts. However, the pulse in FIG. 8 has a substantially greater duration than the pulse in FIG. 7. For example, the long pulse of FIG. 8 can have a duration of approximately 1650 microseconds (250 μs/div*6.6) whereas the short pulse of FIG. 7 can have a duration of about 750 microseconds to about 1000 microseconds. In the illustrated embodiment the areas under each of the pulses shown in FIG. 7 and FIG. 8 are substantially equal. In modified embodiments, however, the areas may vary from one another. Each area may be computed by determining the integral under the voltage trace for each pulse, as understood by persons skilled in the art. Thus, in the illustrated embodiment, the energy of the short pulse and the energy of the long pulse may be substantially equal. By generating pulses of different maximum amplitudes and durations, with for example substantially equal energies in an illustrated embodiment, it may be possible to obtain improvements in cutting, coagulating, or re-modeling of target materials relative to systems which utilize only a single pulse type.

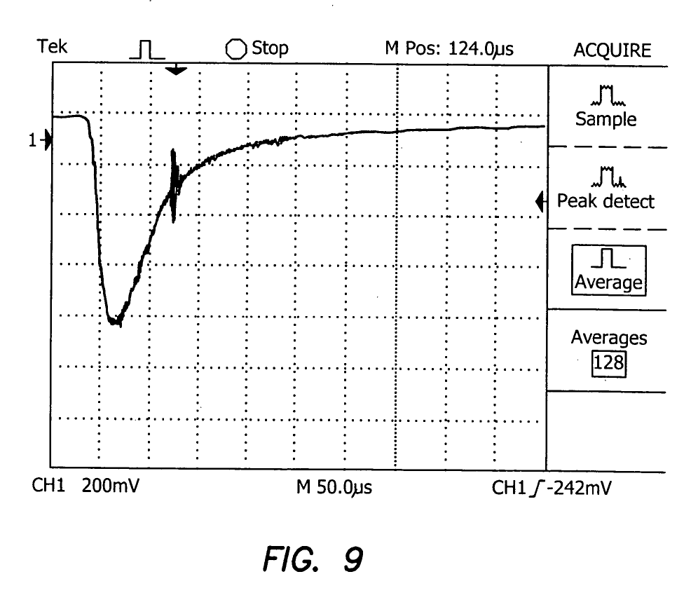

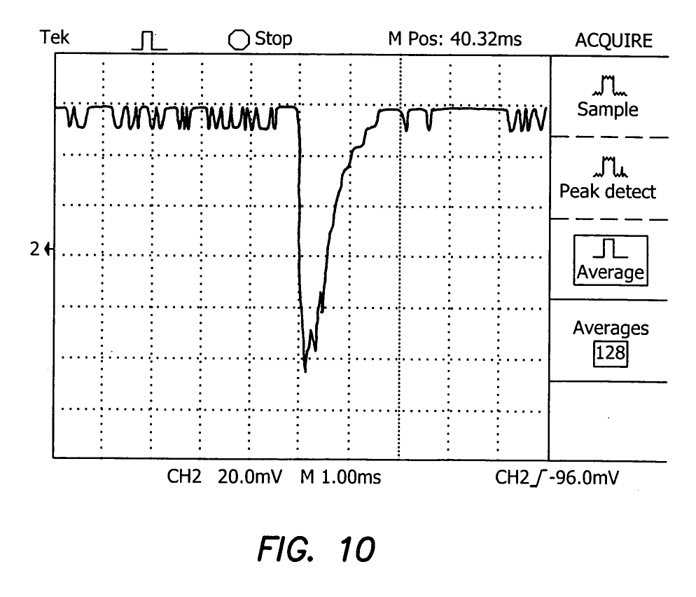

FIGS. 9 and 10 illustrate additional magnified views of a short pulse and a long pulse, respectively. Each voltage trace for the pulse is provided at a different scale.

In certain embodiments, the apparatus disclosed herein may include two high voltage power supplies. In one embodiment, one power supply charges one pulse forming network (e.g., an LC circuit), and the second power supply charges a second pulse forming network (e.g., an LC circuit). Each pulse forming network may then discharge through the same lamp.

The output optical energy distribution 60 of the present invention may be useful for maximizing a cutting effect of an electromagnetic energy source, such as a laser, directed toward a target surface. The cutting and/or ablating effects may occur on or at the target surface, within the target surface, and/or above the target surface. Using the optical energy distributions disclosed herein, it is possible to disrupt a target surface by directing electromagnetic energy toward the target surface so that a portion of the energy is absorbed by fluid. The fluid absorbing the energy may be on the target surface, within the target surface, above the target surface, or a combination thereof. In one embodiment, the fluid absorbing the energy may comprise water and/or may comprise hydroxyl. When the fluid comprises hydroxyl and/or water which highly absorb the electromagnetic energy, these fluid molecules may begin to vibrate. As the molecules vibrate, localized heat is produced that causes expansion leading to disruption (e.g., mechanical, thermo-mechanical, or other types of mechanisms). Other types of disruption effects may occur by the absorption of the impinging electromagnetic energy by other molecules of the target surface. Accordingly, the cutting effects mediated by the energy absorption may be due to thermal properties (e.g., thermal cutting) or thermo-mechanical effects and also by absorptions of the energy by molecules (e.g., water above the target surface) that do not significantly heat the target surface. The use of the electromagnetic energy distributions disclosed herein can reduce secondary damage to the target surface, such as charring or burning, in certain embodiments wherein cutting is performed in combination with a fluid output and also in certain embodiments that do not use a fluid output. Thus, a portion of the cutting effects caused by the electromagnetic energy may be due to thermal energy, and a portion of the cutting effects may be due to disruptive (e.g., mechanical, thermo-mechanical, or other types of effects) forces generated by the disruption of molecules absorbing the electromagnetic energy.

Apparatus used to impart disruptive forces onto a target surface, or cut to coagulate or re-model a target surface, are structured to direct electromagnetic energy toward the target surface so that at least a portion of the energy is absorbed by fluid. One apparatus for imparting disruptive forces onto a target surface is disclosed in U.S. Pat. No. 5,741,247 entitled ATOMIZED FLUID PARTICLES FOR ELECTROMAGNETICALLY INDUCED CUTTING. Not only can the cutting effects of the apparatus be mediated by atomized fluid particles above the target surface, but the cutting effects may alternatively or additionally be mediated by the absorption of energy by fluid on or within the target surface. In one embodiment of the apparatus, the cutting effects are mediated by effects of energy absorption by a combination of fluid located above the target surface, fluid located on the target surface, or fluid located in the target surface. In one embodiment, about one-third of the impinging electromagnetic energy passes through the fluid particles and impinges onto the target surface, and a portion of that impinging energy can operate to cut or contribute to the cutting of the target surface.

A filter may also be provided with the apparatus to modify electromagnetic energy transmitted from the electromagnetic energy source so that the target surface is disrupted in a spatially different manner at one or more points in time compared to electromagnetic energy that is transmitted to a surface without a filter. The spatial and/or temporal distribution of electromagnetic energy may be changed in accordance with the spatial and/or temporal composition of the filter. The filter may comprise, for example, fluid; and in one embodiment the filter is a distribution of atomized fluid particles the characteristics (e.g., size, distribution, velocity, composition) of which can be changed spatially over time to vary the amount of energy impinging on the target surface. As one example, a filter can be intermittently placed over a target to vary the intensity of the impinging energy to thereby provide a type of pulsed effect. In such an example, a spray of water can be intermittently applied to intersect the impinging radiation. In some embodiments, utilization of a filter cutting of the target surface may be achieved with reduced, or no, secondary heating/damage that is typically associated with thermal cutting of prior art lasers that do not have a filter. The fluid of the filter can comprise water. The outputs from the filter, as well as other fluid outputs, energy sources, and other structures and methods disclosed herein, may comprise any of the fluid outputs and other structures/methods described in U.S. Pat. No. 6,231,567, entitled MATERIAL REMOVER AND METHOD, the entire contents of which are incorporated herein by reference to the extent compatible and not mutually exclusive.

The high-intensity leading micropulses 64, 66, and 68 may impart some high peak amounts of energy that are directed toward a target surface. The energy is directed toward the target surface to obtain the desired cutting effects. For example, the energy may be directed into atomized fluid particles and the fluid and/or OH molecules present on or in the material of the target surface which in some instances can comprise water or other bio-compatible fluids, to thereby expand the fluid and induce disruptive (e.g., mechanical) cutting forces to or a disruption (e.g., mechanical disruption, thermo-mechanical or any other types) of the target surface. The trailing micropulses after the maximum micropulse 68 have been found to further help with removal or material. According to the present invention, a single large leading micropulse 68 may be generated or, alternatively, two or more large leading micropulses 68 (or 64, 66, for example) may be generated. In accordance with one aspect of the present invention, relatively steeper slopes of the pulse and shorter pulses may lower the amount of residual heat produced in the material.

The flashlamp current generating circuit 30 of the present invention generates a relatively narrow pulse, which is on the order of 0.25 to 300 microseconds, for example. Additionally, the full-width half-max value of the optical output energy distribution 60 of the present invention can occur within the first 30 to 70 microseconds, for example, compared to full-width half-max values of the prior art occurring within the first 250 to 300 microseconds. The relatively quick frequency, and the relatively large initial distribution of optical energy in the leading portion of each pulse of the present invention, can result in relatively efficient cutting (e.g., mechanical cutting, thermo-mechanical or other types). The output optical energy distributions of the present invention can be adapted for cutting, shaping, removing, coagulating or re-modeling tissues and materials, and further can be adapted for imparting electromagnetic energy into atomized fluid particles over a target surface, or other fluid particles located on or within the target surface. The cutting effect obtained by the output optical energy distributions of the present invention can be both clean and powerful and, additionally, can impart consistent cuts or other disruptive forces onto target surfaces.

The apparatuses disclosed herein may be used to impart cutting forces onto biological and non-biological targets. In most embodiments, the pulse or pulses may be used to generate forces effective to cut, coagulate or re-model body tissues, such as tooth, bone or cartilage. By utilizing trains of varying pulse shapes, as described above, it may be possible to obtain improved or different cutting performances. For example, it may be possible to cut a dental surface with the short, high intensity pulses, and promote closing of dental tubules by a “melting” effect associated with the longer, lower intensity pulses. Such effects may be particularly useful with for example root canal procedures, or for erosion of a tooth or teeth at the gingiva to treat desensitization, such as by closing or melting of tubules to treat desensitization. Alternatively, or in addition, the trains of pulses can be used to fuse re-model dental enamel or dentin, such as to reduce or inhibit cavities. By providing trains of long and short pulses as described above, it may also be possible to cut and coagulate deep tissues, such as vascular tissues.

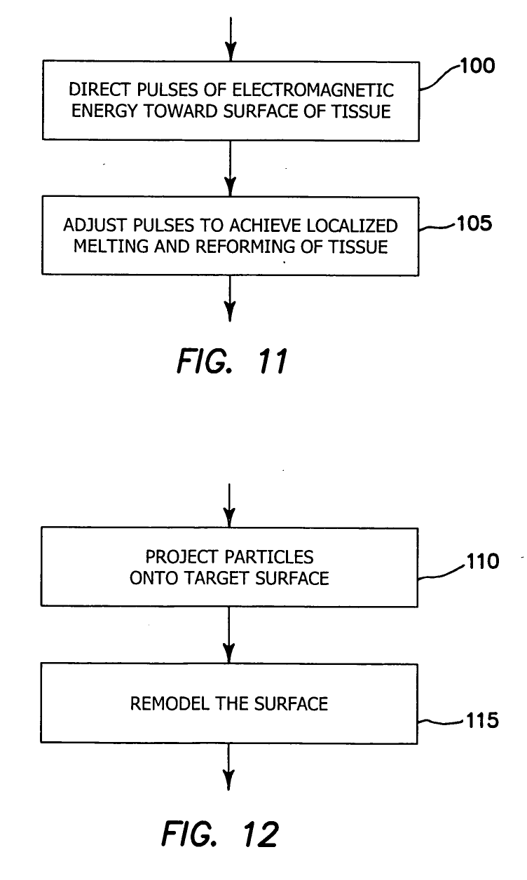

According to another embodiment of the present invention, a fluence of electromagnetic energy (e.g., pulses of electromagnetic energy) directed toward a target (e.g., tissue) may be employed to achieve remodeling of the target. FIG. 11 is a flow diagram describing an implementation of a method of remodeling a target (e.g., tissue) according to the present invention. This implementation comprises directing pulses of electromagnetic energy toward a surface of the tissue at step 100. The tissue, according to one example, may be an upper layer of enamel or dentin tissue of a tooth. Implementation of the exemplary method continues at step 105 by adjusting the pulses to achieve remodeling, which may comprise, for example, localized melting and/or reforming of the tissue and/or other target.

In cases wherein the target comprises hard tissue, remodeling may enhance the hardness of the structure of the tissue, making the tissue more resistant to acid due, for example, to a low pH in the mouth or to acid produced by bacteria. The adjusting may comprise modifying a parameter (e.g., a steepness of a slope or a pulse duration) of an electromagnetic energy pulse as described herein. Generally, pulses having a relatively steep slope as described above may be more effective in certain instances in transferring energy into tissue in order to achieve relatively fast and effective remodeling of, for example, an upper layer of hard tissue. In certain embodiments, while short pulses may be effective in certain implementations for cutting and remodeling, long pulses may be more effective in certain implementations for remodeling. If short pulses are used for cutting, then higher fluences may in certain instances be employed. The adjusting may comprise, for example, varying a duration of various types of pulses, wherein, in one example, the term “duration” is meant to encompass a “full-width half-max range” as described herein and in another example is meant to encompass a pulse length. For example, ultrashort pulses may range in duration from about 0 to 30 μs, short pulses may have durations ranging from about 30 to 150 μs, and long pulses may have durations ranging from about 150 to about 800 μs. Other adjustment techniques may be applied in certain embodiments including, as an example, simultaneous emission of short (or ultrashort) and long pulses. According to another embodiment, pulses may alternate with one type followed by another type, e.g., long and short pulses. Still, other embodiments may alternate trains of pulses wherein a first number of pulses of one type alternates with a second number of pulses of another type. Table 1 lists several examples of types of pulse trains that may be employed. Each entry in the table may represent, for example, an elementary pair of pulses that, in any order (i.e., A+B, or B+A), occurs once and/or repeats or repeats periodically.

TABLE 1

```
  30 μs + 50 μs
  30 μs + 150 μs
  30 μs + 300 μs
  30 μs + 500 μs
  30 μs + 700 μs
  50 μs + 300 μs
  50 μs + 500 μs
  50 μs + 700 μs
150 μs + 300 μs
150 μs + 500 μs
150 μs + 700 μs
```

In remodeling, a thin layer of tissue may be affected (e.g., softened, such as being melted) and allowed to reform (e.g., after cooling). For superficial remodeling, the layer of melted tissue may range from about 0 to 50 μm. For deeper remodeling the melted tissue may range from about 50 to 500 μm Even deeper remodeling, for example up to about 750 μm, may be performed in some embodiments.

It may be important in certain implementations to choose an appropriate (e.g., optimal) thickness that needs to be remodeled so that the remodeling procedure itself does not render the tissue more prone to an acid attack, which may result in for example demineralization or the like. Accordingly, the pulse structure of the applied electromagnetic energy should in accordance with one embodiment be given careful consideration beforehand. Additionally, in applications involving desensitization and caries inhibition, some or all pulse shapes and/or magnitudes may be chosen to stay below a cutting threshold.

For effective remodeling, according to one embodiment, fluence settings may range from about 0.1 J/cm² to about 25 j/cm². In another embodiment, the fluence settings may range from about 0.1 j/cm² to about 10 J/cm². In yet another embodiment, the fluence settings may range from about 0.1 J/cm² to about 5 J/cm². A spot size of about 50 μm to about 1500 μm may be employed in examples of the embodiments.

It should be understood that higher fluence settings may in some implementations result in a greater increase in temperature of treated tissue, e.g., tooth enamel. In order to, for example, reduce a temperature rise under a surface layer of tissue, a procedure to control temperature rise and cool tissue may be performed. In certain implementations, a fluid (e.g., water) and/or air may be applied in order, simultaneously in accordance with any known technique, or in any other combination, for example, to control temperature rise and cool tissue and/or prevent detrimental heat transfer to vital tissues. Generally, in certain implementations, cooling air may be applied to limit a temperature increase, while also, or alternatively, adding in an air/water spray may reduce the temperature of tissue, thereby for example preventing detrimental heat transfer to vital tissue. For example, air alone may be used, directed to the surface at a rate of about 0 to 15 L/min. Alternatively or subsequently, air in combination with water may be used, the air being applied at the same rate and/or the water being applied at a rate of about 0 to 60 ml/min. Generally, for delivery of short pulses, a rate of application of air may range from about 0 to 7 L/min combined with water between about 0 and 10 ml/min according to one embodiment. For delivery of long pulses, the air rate may vary from about 0 to 15 L/min, and the rate of application of water may range from about 0 to 60 ml/min. Application of too much water may in some instances block an effect of a laser on the tissue. Table 2 summarizes an effect of applying air and water as, for example, a cooling technique in an embodiment employing a 2.789 micron Er, Cr:YSGG solid state laser with a 600 μm diameter tip at a distance of about 2 mm from an enamel surface for a time duration of about 10 seconds.

TABLE 2

| Fluence | Air | Water | Temperature |
|---|---|---|---|
| Setting | Rate | Rate | Change |
| 4.4 J/cm² | 0 L/min | 0 ml/min | +7° C. |
| 4.4 J/cm² | 6.9 L/min | 0 ml/min | +2° C. |
| 4.4 J/cm² | 3 L/min | 2.5 ml/min | −2° C. |
| 8.8 J/cm² | 0 L/min | 0 ml/min | +10° C. |
| 8.8 J/cm² | 6.9 L/min | 0 ml/min | +4° C. |
| 8.8 J/cm² | 3 L/min | 2.5 ml/min | +0° C. |

The remodeling technique described herein may be applied in association with, for example, devices and methods for treating (e.g., ablating), for example, dental caries or for desensitization purposes. Examples of such devices and methods are disclosed in U.S. Provisional Patent No. 60/601,415 entitled DUAL PULSE-WIDTH MEDICAL LASER WITH PRESETS, filed Aug. 12, 2004, the entire contents of which are incorporated herein by reference.

Remodeling may be effective to remodel a surface after cavity preparation. Specifically, after tissue has been removed to prepare a cavity, a remodeling procedure may be applied to a last-cut surface. A composite restoration then may be inserted. If, subsequently, leakage occurs, a gap may form between the composite and the cut surface, and bacteria may penetrate into the gap, after which the remodeled surface beneath the restoration may exhibit a reduced tendency for demineralization and secondary decay.

In accordance with another aspect, remodeling may be implemented to inhibit decay formation in areas where decay is likely to develop. For example, pits and fissures on an occlusal surface may be remodeled with small gaps filled by melting and expanding of tissue. In modified embodiments, a sealant may be applied after remodeling. According to another implementation, cervical tooth surfaces may be remodeled to decrease formation of dental caries.

According to implementations of the present invention for prevention of dental caries, fluence settings may range from about 0 J/cm² to about 25 J/cm². In some embodiments, the fluence settings may range from about 0 J/cm² to about 10 J/cm². A spot size of about 50 μm to about 1500 μm may be employed in these embodiments.

Another aspect of the present invention may comprise a method of delivering ions to a target surface. FIG. 12 is a flow diagram summarizing an implementation of this aspect of the present invention. Particles, which may comprise selected types of ions, may be projected onto the target surface at step 110. According to an exemplary embodiment, an air spray, fluid spray or a combination spray of both air and fluid, e.g., biocompatible liquids (e.g. water), may be used to project particles (e.g., ions or ionic compounds) onto the target surface to allow the particles to attach or adhere (e.g., to micromechanically bond) to the surface. The surface then may be remodeled as described herein at step 115, wherein the remodeled tissue layer may be more resistant to caries. The process further may stimulate formation of secondary dentin or cause the surface to exhibit antibacterial properties. According to another aspect of the present invention, a lamination layer may be applied over a target tissue surface so that the tissue surface is laminated with various ionic compounds and then remodeled with a laser. In a modified implementation of the method, the tissue is laminated and remodeled at the same time. Either a wet or dry environment may be employed to implement the layer of ions into the tissue surface.

As examples, ions from a list consisting of fluoride, calcium, phosphorous and hydroxide (OH) may be selected that may enhance caries prevention. As another example, compounds containing ions, e.g., sodium fluoride, stannous fluoride, copper fluoride, titanium tetrafluoride, amine fluorides, calcium hydroxide, hydroxyapatite, calcium phosphate and the like may be selected. It should be noted that some of these compounds may be compatible with soft tissue, and some may be compatible with dentin, enamel, or bone only. More particularly, compounds having a fluoride ion may be effective as anti-caries and desensitizing agents. In accordance with one example, fluoride may act to desensitize dental tissue to effects of, for example, heat and cold. In modified embodiments, compounds including, for example, calcium may aid in forming an anti-bacterial surface. In still further embodiments, remineralization of affected dentin may be enhanced by employing calcium hydroxide or zinc oxide. These compounds may be delivered through water or other biocompatible fluids that contain salt, are sterile, or are low in bacterial count.

The ionic compounds may be applied simultaneously with applying a laser beam, thereby achieving placement of ions and, at the same time, remodeling surface tissue and impregnating the ions into the remodeled layer of tissue. Alternately, the area to be treated first may be sprayed with one or more ion-containing compounds, e.g., a topical fluoride preparation, followed by subsequent application of laser energy.

Table 3 summarizes examples of desired fluoride concentration abstracted from known literature. These concentrations have been noted to be effective in caries prevention.

TABLE 3

| | | |
|---|---|---|
| 250-500 | ppm | Fluoride toothpastes |
| | | May be less effective, but can still be |
| | | used |
| 500-1000 | ppm | |
| 1000-1500 | ppm | Fluoride toothpastes |
| | | Very efficacious |
| 230-920 | ppm | Fluoride mouth rinse |
| 12,300 | ppm | Acidulated phosphate fluoride in |
| | | prescription fluoride gels and foam |
| 9,040 | ppm | Sodium fluoride in prescription fluoride |
| | | foam |
| 5,000 | ppm | Home-use sodium fluoride in fluoride |
| | | gel |
| 1,000 | ppm | Home-use stannous fluoride gel |
| 2,600 | ppm | Fluoride varnish |
| 4,000-20,000 | ppm | Fluoride prophy pastes |

Although an exemplary embodiment of the invention has been shown and described, many other changes, modifications and substitutions, in addition to those set forth in the above paragraphs, may be made by one having ordinary skill in the art without necessarily departing from the spirit and scope of this invention. For example, the methods herein disclosed may be used in the treatment of tooth or bone. A bone-growth inducer such as bone morphogenic proteins may be applied as described herein to help with speeding bone regeneration and repairing bony defects. Any feature or combination of features described herein are included within the scope of the present invention provided that the features included in any such combination are not mutually inconsistent as will be apparent from the context, this specification, and the knowledge of one of ordinary skill in the art.

What is claimed is:

1. An apparatus for imparting disruptive forces onto a target surface, comprising:
(a) an electromagnetic energy source configured to direct electromagnetic energy toward a target surface to impart disruptive forces onto the target surface; and
(b) a flashlamp current generating circuit that generates at least one current pulse to drive the electromagnetic energy source, the current pulses having full-width half-max range positioned substantially within a first half of the current pulse and being shaped to generate electromagnetic energy from the electromagnetic energy source that disrupts the target surface using energy that is absorbed by fluid.

2. The apparatus of claim 1, wherein:
the apparatus is constructed to place fluid on the target surface; and
electromagnetic energy generated by the current pulse is at least partially absorbed by the fluid on the target surface.

3. The apparatus of claim 2, wherein the electromagnetic energy generated by the current pulse is at least partially absorbed by fluid located within the target surface.

4. The apparatus of claim 3, wherein:
the apparatus is constructed to place fluid above the target surface; and
the electromagnetic energy generated by the current pulse is at least partially absorbed by the fluid located above the target surface.

5. The apparatus of claim 4, wherein:
the apparatus is constructed to place the fluid above the target surface as atomized fluid particles; and
electromagnetic energy generated by the current pulse is substantially absorbed by the fluid located above the target surface to impart disruptive forces onto the target surface.

6. The apparatus of claim 3, wherein at least some of the fluid within the target surface that absorbs the electromagnetic energy is not supplied from the apparatus.

7. The apparatus of claim 6, wherein:
the target surface comprises hard or soft tissue; and
the fluid within the target surface comprises water.

8. The apparatus of claim 7, wherein:
the apparatus is constructed to place fluid above the target surface; and
the electromagnetic energy generated by the current pulse is at least partially absorbed by the fluid located above the target surface.

9. The apparatus of claim 8, wherein:
the apparatus is constructed to place the fluid above the target surface as atomized fluid particles;
electromagnetic energy generated by the current pulse is substantially absorbed by the fluid located above the target surface to impart disruptive forces onto the target surface.

10. The apparatus of claim 1, wherein the electromagnetic energy generated by the current pulse is at least partially absorbed by fluid located within the target surface.

11. The apparatus of claim 3, wherein at least some of the fluid within the target surface that absorbs the electromagnetic energy is not supplied from the apparatus.

12. The apparatus of claim 6, wherein:
the target surface comprises hard or soft tissue; and
the fluid within the target surface comprises water.

13. The apparatus of claim 12, wherein:
the apparatus is constructed to place fluid above the target surface; and
the electromagnetic energy generated by the current pulse is at least partially absorbed by the fluid located above the target surface.

14. The apparatus of claim 1, wherein the electromagnetic energy generated by the current pulse is at least partially absorbed by fluid located above the target surface.

15. The apparatus of claim 14, wherein:
the apparatus is constructed to place the fluid above the target surface as atomized fluid particles;
electromagnetic energy generated by the current pulse is substantially absorbed by the fluid located above the target surface to impart disruptive forces onto the target surface.

16. The apparatus of claim 1, wherein the current pulse of the flashlamp current generating circuit, includes:
(i) a leading edge having a slope which is at least 5, the slope being defined on a plot of the pulse as y over x (y/x) where y is current in amps and x is time in microseconds; and
(ii) a full-width half-max value in a range from about 0.025 to about 250 microseconds.

17. The apparatus of claim 1, further comprising a fluid output that outputs fluid between an output of the electromagnetic energy source and the target surface.

18. The apparatus of claim 17, comprising a filter, which comprises fluid that is output from the fluid output, wherein the filter absorbs a portion of the energy generated by the electromagnetic energy source.

19. The apparatus of claim 18, wherein the fluid is atomized particles of water.

20. The apparatus of claim 1, wherein the disruption of the target surface is caused in part by energy generated by the electromagnetic energy source other than the energy absorbed by the fluid.

21. The apparatus of claim 1, wherein the electromagnetic energy source comprises an erbium, yttrium, scandium gallium garnet (Er:YSGG) solid state laser or an erbium, yttrium, aluminum garnet (Er:YAG) solid state laser.

22. A method of imparting disruptive forces onto a target surface, comprising:
(a) positioning an apparatus, which includes an electromagnetic energy source and a flashlamp current generating circuit, in proximity to a target surface so that electromagnetic energy generated by the electromagnetic energy source is capable of being transmitted toward the target surface; and
(b) generating at least one current pulse with the flashlamp current generating circuit, the current pulse having a full-width half-max range positioned substantially within a first half of the current pulse and the current pulse driving the electromagnetic energy source to provide electromagnetic energy that disrupts the target surface by interacting with fluid on or within the target surface.

23. The method of claim 22, further comprising a step of:
(c) filtering the electromagnetic energy with fluid located above the target surface to reduce an intensity of at least a portion of the electromagnetic energy before the portion of electromagnetic energy disrupts the target surface.

24. The method of claim 23, wherein the fluid is provided as a distribution of fluid particles emitted from a fluid output.

25. The method of claim 24, wherein the fluid absorbs a portion of the electromagnetic energy before disrupting the target surface.

26. The method of claim 22, wherein the fluid is water.

27. The method of claim 22, wherein the fluid is disposed within the target surface.

28. The method of claim 22, further comprising a step of:
(c) disrupting the target surface by emitting an atomized distribution of fluid particles from a fluid output of the apparatus above the target surface so that portions of the atomized distribution of fluid particles intersect the electromagnetic energy above the target surface.

29. An apparatus for imparting disruptive forces onto a target surface, comprising:
(a) a laser in communication with a fiberoptic to direct electromagnetic energy from the laser toward the target surface;
(b) a flashlamp current generating circuit that generates at least one current pulse to drive the laser to generate electromagnetic energy from the laser, the current pulse having a full-width half-max range positioned substantially within a first half of the current pulse; and
(c) a filter that is disposed between the fiberoptic and the target surface when electromagnetic energy is transmitted from the fiberoptic, the filter being structured to spatially modify the electromagnetic energy near the target surface so that the target surface is disrupted in a spatially different manner compared to electromagnetic energy that is transmitted to a surface without a filter.

30. The apparatus of claim 29, wherein the filter comprises a distribution of fluid particles that absorb at least a portion of the electromagnetic energy emitted from the fiberoptic.

31. The apparatus of claim 30, wherein the filter comprises spatially distributed fluid particles and the apparatus is constructed to vary the spatial and temporal distributions of the fluid particles.

32. The apparatus of claim 30, wherein the fluid comprises water.

33. The apparatus of claim 29, wherein the flashlamp current generating circuit comprises:
(i) a solid core inductor having a rated inductance of about 50 microhenries;
(ii) a capacitor coupled to the inductor, the capacitor having a capacitance of about 50 microfarads; and
(iii) a flashlamp coupled to the solid core inductor.

34. The apparatus of claim 29, wherein the laser is a Er:YSGG or Er:YAG solid state laser.

35. The apparatus of claim 29, wherein the filter is structured to filter a portion of the electromagnetic energy emitted from the fiberoptic while maintaining the ability of the electromagnetic energy to impart disruptive forces on the target surface by the absorption of energy by fluid on or within the target surface.

36. A method of remodeling tissue, comprising:
directing pulses of electromagnetic energy toward a surface of the tissue; and
adjusting the pulses to achieve localized melting and reforming of the tissue.

37. The method as set forth in claim 36, wherein the melting comprises melting tissue to a depth ranging from about 0 to about 50 μm.

38. The method as set forth in claim 36, wherein the melting comprises melting tissue to a depth ranging from about 50 μm to about 500 μm.

39. The method as set forth in claim 36 wherein the melting comprises melting tissue to a depth not greater than about 750 μm.

40. The method as set forth in claim 36, wherein the adjusting comprises modifying a duration of the pulses.

41. The method as set forth in claim 40, wherein the adjusting further comprises modifying an energy density of the pulses.

42. The method as set forth in claim 41, wherein the modifying of an energy density comprises selecting an energy density ranging from about 0.1 J/cm² to about 25 J/cm².

43. The method as set forth in claim 41, wherein the modifying of an energy density comprises selecting an energy density ranging from about 0.1 J/cm² to about 10 J/cm².

44. The method as set forth in claim 41, wherein the modifying of an energy density comprises selecting an energy density ranging from about 0.1 J/cm² to about 5 J/cm².

45. The method as set forth in claim 40, wherein the modifying comprises selecting a duration from a group comprising ultrashort, short, and long pulses.

46. The method as set forth in claim 45, wherein the selecting of an ultrashort duration comprises selecting a duration ranging from about 0 to about 30 μs.

47. The method as set forth in claim 45, wherein the selecting of a short duration comprises selecting a duration ranging from about 30 μs to about 150 μs.

48. The method as set forth in claim 45, wherein the selecting of a long duration comprises selecting a duration ranging from about 150 μs to about 800 μs.

49. The method as set forth in claim 45, wherein the adjusting comprises simultaneously emitting short and long pulses.

50. The method as set forth in claim 36, further comprising performing a cooling procedure.

51. The method as set forth in claim 50, wherein the performing comprises directing air to the surface.

52. The method as set forth in claim 51, wherein the directing of air comprises directing air at a rate of about 0 to 15 L/min.

53. The method as set forth in claim 50, wherein the performing comprises directing water to the surface.

54. The method as set forth in claim 53, wherein the directing of water comprises directing water at a rate of about 0 to 60 ml/min.

55. The method as set forth in claim 36, wherein dental caries are treated.

56. The method as set forth in claim 36, wherein the remodeling is applied to a surface after cavity preparation.

57. The method as set forth in claim 36, wherein the remodeling inhibits decay formation.

58. The method as set forth in claim 57, wherein the remodeling is applied to an occlusal surface.

59. A method of delivering ions to a target surface, comprising:
projecting particles onto the target surface; and
remodeling the surface.

60. The method as set forth in claim 59, wherein the projecting comprises facilitating micromechanically bonding of the particles to the surface.

61. The method as set forth in claim 59, wherein the projecting comprises employing one of an air spray and a fluid spray to deliver ions to the target surface.

62. The method as set forth in claim 61, wherein the employing of a fluid spray comprises employing a water spray.

63. The method as set forth in claim 59, wherein the projecting comprises employing a combination spray of both air and fluid to deliver ions to the target surface.

64. The method as set forth in claim 63, wherein the projecting comprises projecting particles comprising ions selected from a group comprising fluoride, calcium, phosphorous and hydroxide.

65. The method as set forth in claim 63, wherein the projecting comprises projecting particles comprising compounds containing ions selected from a group comprising sodium fluoride, stannous fluoride, copper fluoride, titanium tetrafluoride, amine fluorides, and calcium hydroxide.

66. The method as set forth in claim 64, wherein the projecting of a fluoride ion inhibits formation of dental caries.

67. The method as set forth in claim 64, wherein the projecting of a fluoride ion desensitizes dental tissue.

68. The method as set forth in claim 64, wherein the projecting of a calcium ion aids in forming an anti-bacterial surface.

69. The method as set forth in claim 64, wherein the projecting of one of a calcium hydroxide and a zinc oxide ion enhances remineralization of dentin.
